(19) United States
(12) Patent Application Publication
(10) Pub. No.: US 2007/0293390 A1
(43) Pub. Date: Dec. 20, 2007

(54) Alkylation Catalyst, Its Preparation and Use

(76) Inventors: Emanuel Hermanus Van Broekhoven, DN Monnickendam (NL); Edgar Evert Steenwinkel, WD Haarlem (NL); Arjan Peter Boomert, KE Alkmaar (NL); Mark Hendrikus Harte, NG Koog aan de Zaan (NL); Danielle Alfos-Gimpel, SP Wormer (NL)

Correspondence Address: Albemarle Netherlands B.V.;Patent and Trademark Department, 451 Florida Street, Baton Rouge, LA, 70801, US

(21) Appl. No.: 11/666,998

(22) PCT Filed: Nov. 3, 2005
(86) PCT No.: PCT/EP05/55740

§ 371 Date: May 3, 2007

(30) Foreign Application Priority Data
Nov. 3, 2004 (EP) 04078024.9

(51) Int. Cl. B01J 37/08; B01J 29/068; B01J 37/30; C07C 2/58; B01J 29/12

(52) U.S. Cl. 502/66; 502/74

(57) ABSTRACT

Process for the preparation of a catalyst comprising the steps of: (a) combining solid acid particles with a binder to form a catalyst precursor; (b) calcining the catalyst precursor at a temperature in the range of about 400 to about 575° C.; (c) impregnating the calcined catalyst precursor with a solution of a Group VIII noble metal and Nh₄⁺ ions, and (d) calcining the impregnated particles at a catalyst temperature in the range of about 400 to about 575° C. The use of two calcination steps in the above temperature ranges results in alkylation catalysts with improved performance.

The present invention relates to a process for the preparation of a catalyst suitable for alkylating a hydrocarbon feed. The invention further relates to the catalyst so obtained, and its use in alkylation processes.

Within the framework of the present invention, the term alkylation refers to the reaction of an alkylatable compound, such as an aromatic or saturated hydrocarbon, with an alkylation agent, such as an olefin. Without limiting the scope of the invention, we will further illustrate the invention by discussing the alkylation of saturated hydrocarbons, in general branched saturated hydrocarbon, with an olefin to give highly branched saturated hydrocarbons with a higher molecular weight. Hydrocarbons contain no atoms other than hydrogen and carbon. This reaction is of interest because it makes it possible to obtain, through the alkylation of isobutane with an olefin containing 2 to 6 carbon atoms, an alkylate which has a high octane number and which boils in the gasoline range. Unlike gasoline obtained by cracking heavier petroleum fractions such as vacuum gas oil and atmospheric residue, gasoline obtained by alkylation is essentially free of contaminants such as sulfur and nitrogen and so has clean burning characteristics. Its high anti-knock properties, represented by the high octane number, lessen the need to add environmentally harmful anti-knock compounds such as aromatics or lead. Also, unlike gasoline obtained by reforming naphtha or by cracking heavier petroleum fractions, alkylate contains few if any aromatics or olefins, which, environmentally speaking, is a further advantage.

The alkylation reaction is acid-catalysed. At present, in commercial alkylation equipment use is made of liquid acid catalysts such as sulfuric acid and hydrogen fluoride. The use of such catalysts is attended with a wide range of problems. For instance, sulfuric acid and hydrogen fluoride are highly corrosive, so that the equipment used has to meet high quality requirements. Since the presence of highly corrosive materials in the resulting fuel is objectionable, the remaining acid has to be removed from the alkylate. Also, because of the phase separations that have to be carried out, the process is complicated and thus expensive. Besides, there is always the risk that toxic substances such as hydrogen fluoride will be emitted.

A more recent development in this field is the use of solid acid catalysts, such as zeolite-containing catalysts. WO 98/23560 discloses the use in the alkylation of hydrocarbons of a catalyst containing a zeolite, such as a Y zeolite, a Group VIII noble metal (e.g., platinum or palladium) as hydrogenation component, and optionally a matrix material, such as alumina.

Such a catalyst can be prepared by mixing the solid acid with matrix material, shaping the mixture to form particles, and calcining the particles. The hydrogenation component may be incorporated into the catalyst composition by impregnation of said particles.

EP 1 308 207 discloses an alkylation process using a catalyst comprising a solid acid, a hydrogenation component consisting essentially of one or more Group VIII noble metals, and at least 0.05 wt % of sulfur. This catalyst is prepared by contacting a material comprising the solid acid and the hydrogenation component with a sulfur-containing compound.

This document discloses different methods for preparing the material comprising the solid acid and the hydrogenation component, one of these methods involving the steps of:
(i) shaping, e.g. extruding, the solid acid, optionally after mixing it with a matrix material, to form particles;
(ii) calcining the resulting particles, and
(iii) incorporating the hydrogenation component into the calcined particles by, e.g., impregnating the particles with a solution of one or more Group VIII noble metals and/or by (competitive) ion exchange.

The so-prepared material is preferably calcined and reduced prior to its contact with the sulfur-containing compound.

It has now been found that the performance in alkylation reactions of noble metal-containing solid acid catalysts can be further improved if the calcination steps before and after incorporation of the hydrogenation component—i.e. steps a) and d) mentioned below—are both conducted in a specific catalyst temperature window.

The present invention therefore relates to a process for the preparation of a catalyst comprising the steps of:
(a) combining solid-acid particles with an alumina binder to form a catalyst precursor;
(b) calcining the catalyst precursor at a catalyst temperature in the range of about 400 to about 575° C.;
(c) impregnating the calcined catalyst precursor with a solution of a Group VIII noble metal compound, said solution further comprising NH₄⁺ ions, and
(d) calcining the impregnated catalyst precursor obtained in step c) at a catalyst temperature in the range of about 400 to about 500° C.

As illustrated by the Examples below, it is important that the temperature during both the first and the second calcination step is in the claimed temperature window.

The Solid Acid-Containing Particles

The solid acid-containing particles generally comprise a solid acid and a matrix material.

Examples of suitable solid acids are zeolites such as zeolite beta, MCM-22, MCM- 36, mordenite, X-zeolites and Y-zeolites, including H-Y-zeolites and USY-zeolites, non-zeolitic solid acids such as silica-alumina, sulfated oxides such as sulfated oxides of zirconium, titanium, or tin, mixed oxides of zirconium, molybdenum, tungsten, phosphorus, etc., and chlorinated aluminium oxides or clays. Preferred solid acids are zeolites, including mordenite, zeolite beta, X-zeolites and Y-zeolites, the latter including H-Y-zeolites and USY-zeolites. Mixtures of solid acids can also be employed. The X- and Y-zeolites may also be exchanged with multivalent cations, such as (mixtures of) rare earth ions. An even more preferred solid acid is Y-zeolite with a unit cell size of 24.34-24.72 angstroms, and most preferred is a Y-zeolite with a unit cell size of 24.42-24.56 angstroms.

Examples of suitable matrix materials are alumina, silica, titania, zirconia, clays, and mixtures thereof. Matrix materials comprising alumina are generally preferred.

Preferably, the solid acid-containing particles comprise from about 2 to about 98 wt % of the solid acid and from about 98 to about 2 wt % of the matrix material, based on the total weight of the solid acid and the matrix material present in the particles. More preferably, the solid acid-containing particles comprise from about 10 to about 90 wt % of the solid acid and from about 90 to about 10 wt % of the matrix material. Even more preferably, the solid acid-containing particles comprise from about 10 to about 80 wt % of the matrix material and balance solid acid, most preferably they comprise from about 10 to about 40 wt % of the matrix material and balance solid acid, based on the total weight of the solid acid and the matrix material contained in the particles.

The solid acid-containing particles can be prepared by standard methods, e.g. mixing a solid acid and a matrix material and shaping the mixture to form shaped bodies. A preferred shaping method is extrusion, but also agglomeration, spray drying, and beads formation by, e.g., the oil droplet method can be used. Suitable shapes of said particles include spheres, cylinders, rings, and symmetric or asymmetric polylobes, for instance tri- and quadrulobes. Preferably, the catalyst particles have an average particle diameter of at least about 0.5 mm, more preferably of at least about 0.8 mm, and most preferably of at least about 1.0 mm. The upper limit of the average particle diameter preferably lies at about 10.0 mm, more preferably at about 5.0 mm, even more preferably at about 3.0 mm.

Step a)

The solid-acid particles are combined with a binder material to form a catalyst precursor. Binder materials are well known in the art, and may comprise silica, alumina, or silica/alumina. For preparation of the catalyst of the present invention alumina is the preferred binder material.

Step b)

The catalyst precursor is calcined at a temperature in the range of from about 400 to about 575° C., preferably From about 450 to about 550° C., more preferably from about 460 to about 500° C.

The heating rate preferably ranges from about 0.1 to about 100° C./min, more preferably from about 0.5° C. to about 50° C./min, most preferably from about 1 to about 30° C./min.

Calcination is preferably conducted for about 0.01 to about 10 hrs, more preferably from about 0.1 to about 5 hrs, most preferably from about 0.3 to about 2 hrs.

It is preferably conducted in an air and/or inert gas (e.g. nitrogen) flow. More preferably, this atmosphere is dry.

Preferably, the catalyst precursor is dried before being calcined. This drying is preferably conducted at a temperature of from about 110 to about 150° C.

The calcination can be performed in any equipment, such as a fixed bed reactor, a fluidized bed calciner, and a rotating tube calciner.

Step c)

A Group VIII noble metal is then incorporated into the calcined solid acid-containing particles. This is preferably done by impregnation or competitive ion exchange of the solid acid-containing particles using a solution comprising Group VIII noble metal ions and/or their complexes and NH₄⁺ ions. Preferred Group VIII noble metals are platinum, palladium, and combinations thereof. More preferably, at least one of the Group VIII noble metals is platinum.

Suitable Group VIII noble metal salts include nitrates, chlorides, and ammonium nitrates of the noble metals or their complexes (e.g. NH₃ complexes).

Step d)

The resulting noble metal-containing particles are then calcined at a temperature in the range of from about 400 to about 500° C., preferably from about 450 to about 500° C. It is important to calcine at a temperature of at least about 400° C. to remove substantially all nitrogen compounds that were introduced during impregnation. It has been found that the presence of nitrogen compounds in the catalyst negatively affects the catalyst's performance.

This temperature is preferably reached by heating the particles by about 0.1 to about 100° C./min, more preferably from about 0.5 to about 50° C./min, most preferably from about 1 to about 30° C./min to the desired final value between from about 400 to about 500° C.

Calcination is preferably conducted for about 0.01 to about 10 hrs, more preferably from about 0.1 to about 5 hrs, most preferably from about 0.3 to about 2 hrs.

Calcination is preferably conducted in an air and/or inert gas (e.g. nitrogen) flow. More preferably, this atmosphere is dry.

Optionally, a separate drying step is applied between steps (c) and (d). Alternatively, the noble metal-containing particles are dried during the calcination step.

Also optionally, a dwell of about 15 to about 120 minutes, preferably from about 30 to about 60 minutes is introduced at a temperature of about 200 to about 250° C.

After calcination step (d), the resulting catalyst particles are preferably reduced at a preferred temperature range of from about 200 to about 500° C., more preferably from about 250 to about 350° C., in a reducing gas such as hydrogen

Before or after this reduction treatment, water may be added to the catalyst particles. As described in non-prepublished European Patent Application No. 04075387.3, the presence of 1.5-6 wt % of water, more preferably 1.8-4, and most preferably 2-3 wt %—measured as the loss on ignition at 600° C.—has a positive effect on the alkylation activity and the alkylate quality.

The Alkylation Process

Preferably, the hydrocarbon to be alkylated in the alkylation process is a branched saturated hydrocarbon such as an isoalkane having 4 to 10 carbon atoms. Examples are isobutane, isopentane, isohexane or mixtures thereof, with isobutane being most preferred. The alkylation agent preferably is an olefin having 2 to 10 carbon atoms, more preferably 2 to 6 carbon atoms, still more preferably 3 to 5 carbon atoms, and most preferably 4 carbon atoms. Most preferably, the alkylation process consists of the alkylation of isobutane with butenes.

As will be evident to the skilled person, the alkylation process can take any suitable form, including fluidised bed processes, slurry processes, and fixed bed processes. The process can be carried out in a number of beds and/or reactors, each with separate addition of alkylation agent if desirable. In such a case, the process of the invention can be carried out in each separate bed or reactor.

Suitable process conditions are known to the skilled person. Preferably, an alkylation process as disclosed in WO 98/23560 is applied. In this process the catalyst is subjected intermittently to a mild regeneration step by being contacted with a feed containing a saturated hydrocarbon and hydrogen. This mild regeneration is preferably carried out at about 90% or less of the active cycle of the catalyst, whereby the active cycle is defined as the time from the start of the feeding of the alkylation agent to the moment when, in comparison with the entrance of the catalyst-containing reactor section, about 20% of the alkylation agent leaves the catalyst-containing reactor section without being converted, not counting isomerisation inside the molecule.

The process conditions applied in the present process are summarised in the following Table:

| | | | Molar ratio |
|---|---|---|---|
| | Temperature | Pressure | of hydrocarbon |
| | range of [° C.] | range of {bar] | to alkylation agent |
| Preferred | −40-250 | 1-100 | 5:1-5,000:1 |
| More preferred | 20-150 | 5-40 | 50:1-1,000:1 |
| Most preferred | 65-95 | 15-30 | 150:1-750:1 |

The mild regeneration is preferably conducted at temperatures and pressures that differ from the reaction temperature by not more than about 50%, more preferably by not more than about 20%, still more preferably by not more than about 10%.

Optionally, in the above process the catalyst particles may be subjected to a high-temperature regeneration with hydrogen in the gas phase. This high-temperature regeneration is preferably carried out at a temperature of at least about 150° C., more preferably at about 150 to about 600° C., and most preferably at about 200 to about 400° C. For details of this regeneration procedure, reference is made to WO 98/23560. The high-temperature regeneration can be applied periodically during the alkylation process and is preferably applied after about every 50, more preferably after about every 100, and most preferably after about every 200 to about 400 mild regenerations.

If as a result of high-temperature regeneration the water content of the catalyst particles has decreased to below the desired level, the catalyst particles may be rehydrated during the process in the ways described in the patent application cited above.

Preferably, in addition to the high-temperature regeneration treatment a milder regeneration is applied during the alkylation process, such as described in WO 98/23560, in particular page 9, line 13 through page 13, line 2. This text passage is incorporated herein by reference. More in particular, during the alkylation process the catalyst particles are preferably subjected intermittently to a regeneration step by being contacted with a feed containing a hydrocarbon and hydrogen, with said regeneration preferably being carried out at about 90% or less, more preferably at about 60% or less, even more preferably at about 20% or less, and most preferably at about 10% or less of the active cycle of the catalyst. The active cycle of the catalyst is defined as the time from the start of the feeding of the alkylation agent to the moment when, in comparison with the alkylation agent added to the catalyst-containing reactor section, about 20% of the alkylation agent leaves the catalyst-containing reactor section without being converted, not counting isomerisation inside the molecule

The quality of the alkylate product obtained in the process according to the invention can be measured by its Research Octane Number (RON). The RON is a measure of the anti-knock rating of gasoline and/or gasoline constituents. The higher the RON, the more favorable the anti-knock rating of the gasoline will be. Depending on the type of gasoline engine, generally speaking a higher anti-knock rating is of advantage when it comes to the working of the engine. The product obtained in the process according to the invention preferably has a RON of about 90 or higher, more preferably of about 92 or higher, most preferably about 94 or higher. The RON is obtained by determining, e.g. via gas chromatography, the percentages by volume of the various hydrocarbons in the product. The percentages by volume are then multiplied by the RON contribution and added up.

Examples of compounds with a RON of 90 or higher are isopentane, 2,2-dimethyl butane, 2,3-dimethyl butane, trimethyl butane, 2,3-dimethyl pentane, 2,2,4-trimethyl pentane, 2,2,3-trimethyl pentane, 2,3,4-trimethyl pentane, 2,3,3-trimethyl pentane, and 2,2,5-trimethyl hexane.

### EXAMPLES

### Examples 1-6

Dried extrudates comprising 70 wt % of USY-zeolite and 30 wt % of an alumina matrix were calcined in air at different final temperatures for about 1 hour after being heated at a rate of about 5° C./min. The calcination temperatures applied are listed in Table 1 as “T calcination 1”.

The calcined extrudates were subsequently impregnated with an aqueous solution of Pt(NH₃)₄Cl₂ and NH₄NO₃ by incipient wetness. The amount of NH₄+ ions was equivalent to the number of Na+-exchangeable sites of the catalyst. After drying at 120° C. for 2 hours the impregnated extrudates were calcined for 2 hours in air at different final catalyst temperatures (“T calcination 2” in Table 1) to obtain catalyst particles. The heating rate to the final temperature was about 5° C./min (with a dwell of about 2 hrs at 230° C.).

The resulting Pt-content of the extrudates was 0.34 wt %.

These catalyst particles were subsequently tested according to the following procedure.

A fixed-bed recycle reactor as described in WO 98/23560 having a diameter of 2 cm was filled with a 1:1 volume/volume mixture of 38.6 grams of catalyst extrudates (wetted in ambient air to a Loss On Ignition (600° C.) of about 4.5 wt %) and carborundum particles (60 mesh). At the centre of the reactor tube a thermocouple of 6 mm in diameter was arranged. The reactor was flushed with nitrogen for 30 minutes (21 Nl/hour). Next, the system was tested for leakages at elevated pressure, after which the pressure was raised to 21 bar and the nitrogen replaced by hydrogen (21 Nl/hour). The reactor temperature was then raised to 275° C. at a rate of 1° C./min and the catalyst was reduced at 275° C. After 2 hours, the reactor temperature was lowered to the reaction temperature.

The hydrogen stream was stopped with the attaining of the reaction temperature. Isobutane was supplied to the reactor at a rate of about 4,000 grams/hour. About 95-98% of the isobutane was fed back to the reactor. About 2-5% was drained off for analysis. Such an amount of isobutane was supplied to the reactor as to ensure a constant quantity of liquid in the system. When the system had stabilized, such an amount of cis-2-butene was added to it as to give a cis-2-butene-WHSV of 0.19 (calculated on zeolite weight in the catalyst sample). The overall rate of flow of liquid in the system was maintained at about 4,000 g/h. The weight ratio of isobutane to cis-2-butene at the reactor inlet was about 750. The pressure in the reactor amounted to 21 bar.

Each time after 1 hour of reaction, the catalyst particles were regenerated by being washed with isobutane for 5 minutes, followed by 50 minutes of regeneration through being contacted with a solution of 1 mole% of H2 in isobutane, and then being washed with isobutane for another 5 minutes (total washing and regeneration time 1 hour). After this washing step, alkylation was started again. The temperature during the washing steps, the regeneration step, and the reaction step was the same.

After processing as above for 24 hours at the same temperature, a pseudo-steady state was reached. Then, the temperature was decreased and the process was conducted as above for another 24 hours. Hence, the catalytic performance was measured at various temperatures going from higher to lower.

The performance was characterized by the reaction temperature and the research octane number (RON) at 99.5% olefin conversion per reactor pass. The RON was determined as described on pages 13 and 14 of WO 9823560, the only exception being that the RON contribution of total C9+ (excl. 2,2,5-trimethylhexane) was estimated to be 84 instead of 90. The C5+ alkylate yield is defined as the weight amount of C5+ alkylate produced divided by the overall weight of olefin consumed.

The effect of the calcination temperatures on the performance of the catalyst particles is indicated in Table 1:

TABLE 1

| | | | Reaction T | |
|---|---|---|---|---|
| | T calcination 1 | T calcination 2 | at 99.5% | RON at |
| Example | (° C.) | (° C.) | conv. (° C.) | 99.5% conv. |
| 1 | 450 | 450 | 61 | 96.7 |
| 2 | 475 | 450 | 52 | 97.3 |
| 3 | 500 | 450 | 57 | 97.3 |
| 4 | 540 | 450 | 58 | 97.2 |
| 5 | 500 | 600 | 60 | 97.0 |
| 6 | 600 | 450 | 64 | 96.6 |

Table 1 clearly shows that the performance of the alkylation catalyst can be optimised by varying the calcination temperature both before and after impregnation. Application of calcination temperatures in the claimed range results in improved alkylation catalysts.

### Examples 7-8

A catalyst was prepared as in Examples 1-6 using 475° C. for T calcination 1. After impregnation a sample of this catalyst was calcined at a catalyst temperature of 350° C. (Example 7). A second sample of this catalyst was calcined at a catalyst temperature of 450° C. (Example 8). Both samples were analysed for residual nitrogen. The nitrogen content of the catalyst of Example 7 was 408 ppm. The nitrogen content of the catalyst of Example 8 was <30 ppm.

The catalyst of Example 7 showed 99.5% conversion at 55° C. and RON at 99.5% conversion of 97.2.

The catalyst of Example 8 showed 99.5% conversion at 52° C. and RON at 99.5% conversion of 97.5.

1. A process for the preparation of a catalyst which is suitable for alkylating a hydrocarbon feed, said process comprising the steps of:
a) combining particles of a solid-acid selected from the group consisting of zeolite betz, MCM-22, MCM-36, mordenite, X-zeolites, Y-zeolites, and mixtures thereof with a binder material to form a catalyst precursor;
b) calcining the catalyst precursor at a temperature in the range of from about 400 to about 575° C.;
c) impregnating the calcined catalyst precursor with a solution of a Group VIII noble metal compound, said solution further comprising NH₄⁺ ions, and
d) calcining the impregnated catalyst precursor obtained in step c) in air and/or inert gas at a temperature in the range of about 400 to about 500° C.

2. The process according to claim 1 wherein the temperature applied in step a) is in the range of about 450 to about 550° C. 450-550° C.

3. The process according to claim 2 wherein the temperature in step a) is in the range of about 460 to about 500° C.

4. The process according to claim 1 wherein the temperature in step d) is in the range of about 450 to about 500° C.

5. The process according to claim 1 wherein the solid acid is a zeolite selected from the group consisting of mordenite, zeolite beta, X- zeolites, and Y-zeolites.

6. The process according claim 1 wherein the binder is alumina.

7. A catalyst suitable for alkylating a hydrocarbon feed, wherein the catalyst is prepared by:
a) combining particles of a solid-acid selected from the group consisting of zeolite beta, MCM-22, MCM-36, mordenite, X zeolites, Y zeolites, and mixtures thereof with a binder material to form a catalyst precursor,
b) calcining the catalyst precursor at a temperature in the range of from about 400 to about 575° C.;
c) impregnating the calcined catalyst precursor with a solution of a Group VIII noble metal compound and NH₄⁺ ions, and
d) calcining the impregnated catalyst precursor obtained in step c) in air and/or inert gas at a temperature in the range of about 400 to about 500° C.

8. (canceled)

9. (canceled)
